(19) United States
(12) Patent Application Publication
(10) Pub. No.: US 2020/0243160 A1
(43) Pub. Date: Jul. 30, 2020

(54) Method and Apparatus for Performing a Normalization in the Context of Sequencing Analysis

(71) Applicant: Qiagen GmbH, Hilden (DE)

(72) Inventors: Fernando OESTERREICH CARRILLO, Düsseldorf (DE); MAIKO LOHEL, Hilden (DE); THORSTEN ZERFASS, Mühleim an der Ruhr (DE)

(21) Appl. No.: 16/624,661

(22) PCT Filed: Jun. 19, 2018
(86) PCT No.: PCT/EP2018/066278

§ 371 Date: Dec. 19, 2019

(30) Foreign Application Priority Data
Jun. 20, 2017 (EP) 17177001.9

(51) Int. Cl. G16B 30/00; G16B 45/00; C12Q 1/6869; G06F 17/17; G06T 7/00

(52) U.S. Cl. CPC G16B 30/00; G16B 45/00; G06T 2207/30072; G06F 17/17; G06T 7/0012; C12Q 1/6869

(57) ABSTRACT

A method for performing a normalization of intensity values obtained to perform sequencing analysis comprises the steps: receiving (110) a plurality of image data for a plurality of channels, each image data (11a-11d, 15a-15d) describes for the respective channel (a-d) an intensity distribution over all positions of an image of the a plurality of image data (11 a-11 d, 15a-15d); parametrization (120) of the intensity distribution over all positions for the plurality of channels (a-d) to obtain parametrized distributions for the plurality of channels (a-d); combining (130) the parametrized distributions for the plurality of channels (a-d) to obtain a common distribution for all of the plurality of channels (a-d); and determining (140) for each of the plurality of channels (a-d) a transfer function such that the respective transfer function for the respective channel (a-d) maps the corresponding intensity distribution to the common distribution.

Embodiments of the present invention refer to a method for performing the normalization of the intensity values obtained to perform sequencing analysis. Additional embodiments refer to a corresponding computer program and the corresponding apparatus.

Next generation sequencing (NGS) characterizes the sequence of millions of DNA molecules in parallel. To this end, millions of DNA molecules are immobilized randomly at different positions on an imaging surface and copied to form local clusters of clonal DNA molecules. Sequencing of these template molecules is performed by synthesis of complementary DNA, incorporating fluorescently labeled nucleotides with 4 distinct fluorescent dyes for each specific nucleotide (A, C, G, and T). Specific sequencing chemistry ensures that each sequencing iteration (cycle) incorporates only one nucleotide at a time. For each cycle the sequencing apparatus (sequencer) takes images with four distinct filter settings (channels), one for each nucleotide specified wavelength specified by the respective fluorescent dyes. The entire sequencing run is therefore represented by a set of n×c images, where n equals the number of channels (typically 4) and c equals the number of cycles.

This set-up enables deduction of the nucleotide-sequence of the template DNA molecules from the sequence of images acquired for all channels for each sequencing cycle: Given that the position of a template DNA cluster is known, the intensity profiles over all channels at this given position allows deduction of the incorporated fluorescently labeled nucleotide (base-calling). In theory non-zero intensities should be detectable in only one channel which resembles the nucleotide present in the template DNA. A variety of factors including imaging and sequencing noise result in a derivation from this optimal case: non-zero intensity values are typically observed for all channels. Performing base-calling for all sequencing cycles for a given position allows deduction of the full nucleotide sequence (read).

As indicated above, the signal distribution among the channels determines base-calling. However, deviations from the optimal case (i.e. only one channel is characterized by non-zero intensity values) can be ascribed to two components:
i) bias; and
ii) noise.

While the first can be described as a systematic offset from zero, the latter can be described by fluctuation around this offset from sample to sample. Importantly, these components can be channel-specific and thus may lead to biased base-calling. Several channel-specific bias introducing phenomena are known and are typically corrected in the algorithm chain leading to base-calling. These include background correction and crosstalk correction. However, only known phenomena can be corrected using such a model based approach. Therefore, there is the need for an improved approach.

It is an objective of the present invention to provide a concept for base-calling or especially the post image analysis used for base-calling procedures having a reduced impact to biasing and noise effects.

This objective is solved by the subject matter of the independent claims.

Embodiments of the present invention provide a method for performing a normalization of intensity values obtained to perform a sequencing analysis. The method comprises the four basic steps of “receiving a plurality of image data for a plurality of channels”, “parametrization of an intensity distribution over all or a subset of all positions for the plurality of channels”, “combining parametrized distributions for the plurality of channels” and determining for each of the plurality of channels a transfer function”. The image data comprise a plurality of images (e.g. generated by a sequencing apparatus). Each received image describes for the respective channel, for example for the four channels A, C, G, and T (belonging to four bases), an intensity distribution in terms of a spatial light density distribution over all positions or at least over a subset of all positions of the respective image. The intensity distribution is parametrized to obtain a parametrized distribution for each of the plurality of channels. Starting from the parametrized distributions for the plurality of channels, the parametrized distributions are combined with the aim to obtain a common distribution for all or for at least two of the plurality of channels. The last step of determining the transfer function for each channel is performed such that the respective transfer function for the respective channel maps the corresponding intensity distribution to the common distribution.

Teachings disclosed herein are based on the finding that the intensity distribution is typically channel-specific leading to systematic differences between the channels. However, since over the entire image, the number of A-, C-, G-, and T-base-calls should be randomly equally distributed, the overall intensity level of all channels should be comparable to each other. In order to maintain the comparableness, all or the relevant channels can be adapted with regard to its intensity level. This is achieved when collapsing each channel-specific intensity distribution into one common distribution and determining corresponding correction factors for the respective channel such that same can be mapped to the (collapsed) common distribution, afterwards. The usage of the correction factor for the respective channel produces unbiased intensity profiles over all channels and leading to unbiased base-calling (without or with reduced systematic differences). Here, it is beneficial that the correction of the base-calling can be performed without knowing the exact phenomenon leading to the biasing.

According to another embodiment, the step of parametrization is performed using the substep of maximum-likelihood estimation, maximum-a-posteriori-estimation, determining a summary statistic for one or more or all channels or determining a specific parameter set describing an intensity distribution (e.g. the maxima and minima of said distribution) for one or more or all channels. The estimation for determining procedures enables beneficially to parametrize different intensity distributions, such that the same are comparable to each other.

According to embodiments a distinction between two types of intensity distributions is made, namely non-normal distributions and normal distributions. Commonly, the type of intensity distribution has an impact to the type of the transfer function, so that typically, a non-linear transformation is used for non-normal distributions, wherein a linear transformation is used for normal distributions. For example, the parametrized distribution for one or more or all of a plurality of channels is described by a Gaussian distribution by a distinct mean and a distinct standard deviation. Here, the respective transfer function for the respective channel can also be described by a Gaussian distribution comprising the mathematic operations for subtracting the mean from the corresponding intensity distribution and/or dividing the corresponding intensity distribution by the standard deviation. According to another embodiment transfer functions like a log-transformation, a square-root transformation, a Box-Cox transformation or a Yeo-Johnson transformation can be used for non-normal intensity distributions. Alternatively, another transformation enabling to transform non-normal distribution to an approximated normal distribution can be applied. Since the intensity distributions for different sequencing analysis techniques or procedures vary with regard to its type, it is beneficial to have different transformation approaches enabling to handle the different types of intensity distributions such that the normalization process can be applied to each used case.

Although the above embodiments suggest performing the normalization over the different channels within one cycle of a sequencing analysis, it should be noted that according to further embodiments the parametrization, combining and determining is performed for a plurality of cycles. In this case, the method comprises the step of determining the transfer functions such that same comprise smoothing functions. Here, smoothing means that the transfer function smoothes the intensity distribution over the multiple cycles such that there are no jumps from cycle to cycle, i.e. the smoothing function is selected such that same describes a maximum change of the intensity value over the number of cycles and/or such that the intensity value within at least two subsequent cycles remain on a constant level. This case depends on the assumption that channel-specific distributions of subsequent cycles are similar and thus their characterization parameters (i.e. distribution parameters or summary statistics) are correlated. According to another variant the normalization over a plurality of cycles may be performed such that a normalization over all cycles of the analysis can be done. Here, the respective transfer function for the respective channels comprise a normalization function enabling that the intensity values of the plurality options are normalized over the number of cycles for all channels and/or such that the intensity values remain on an average constant level over the number of cycles. This approach enables to correct effects like an intensity drift. However, in case there is a trend which causes decreasing or increasing intensity values of the number of cycles this approach may have a negative effect to the sequencing analysis. Therefore, the normalization function is determined just in the case if no Trend of the one or more channels over the number of cycles is expected or detected. This trend detection may be performed based on the image data before applying the transfer function to the plurality of channels and cycles.

Of cause, it is possible to perform the normalization (parametrization, combining and determining of the common target distribution) over the different channels and different cycles. Thus, according to another embodiment the basic method may be performed differently, namely such that the step of receiving a plurality of image data and prioritization of the intensity distribution is performed for a plurality of channels and cycles, wherein the step of combining and determining is performed for the plurality of cycles. Expressed in other words this means that the transfer function enables a mapping over a plurality of cycles instead of a mapping over a plurality of channels. Here, the principles as discussed in context of this moving function may be applied for at least one channel. As described above, the combination of normalizing the intensity distribution over cycles and channels is also possible.

The above embodiments start from the assumption that preferably (but not necessarily) all of the channels are mapped to a common distribution function. However, especially in case when a smoothing over a plurality of cycles is performed, it may be beneficial that a common distribution function is determined for each channel or for at least two channels independently. Here, the common distribution function for all of the plurality of channels may be described by a set of functions comprising for each channel a common distribution function, wherein at least two of the common distribution functions differ from each other. Each respective transfer function is determined such that the respective transfer function for a respective channel maps the corresponding intensity distribution to the corresponding common distribution function of the channel. As mentioned above, this approach having a plurality of common distribution functions is selected, if a smoothing over a plurality of cycles is performed. Here, the plurality of common distribution functions may be used beneficially if a trend of one or more channels over the number of cycles is expected or detected.

According to another embodiment the computer program having a program code for performing one of the above methods or method steps.

According to another embodiment and an apparatus for performing a normalization of intensity values obtained to perform a sequencing analysis is provided. Here, the apparatus comprises an interface and a processor. The interface receives the plurality of image data, wherein the processor performs the parametrization, combination and determining of the transfer functions.

Embodiments of the present invention will subsequently be discussed referring to the enclosed figures, wherein:

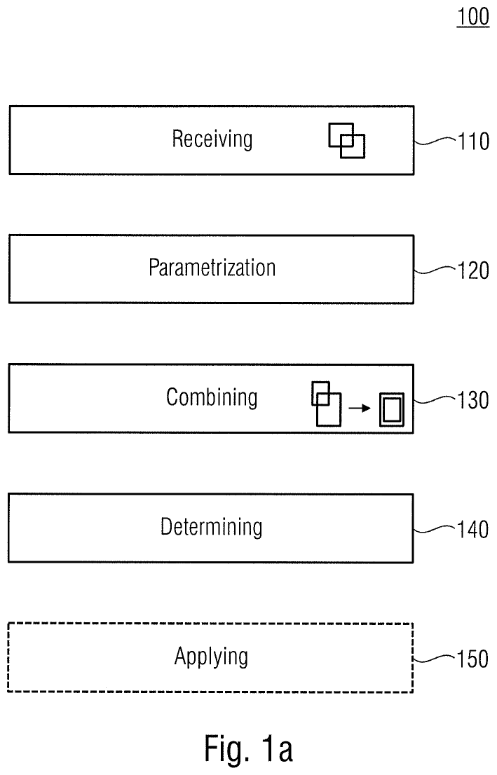

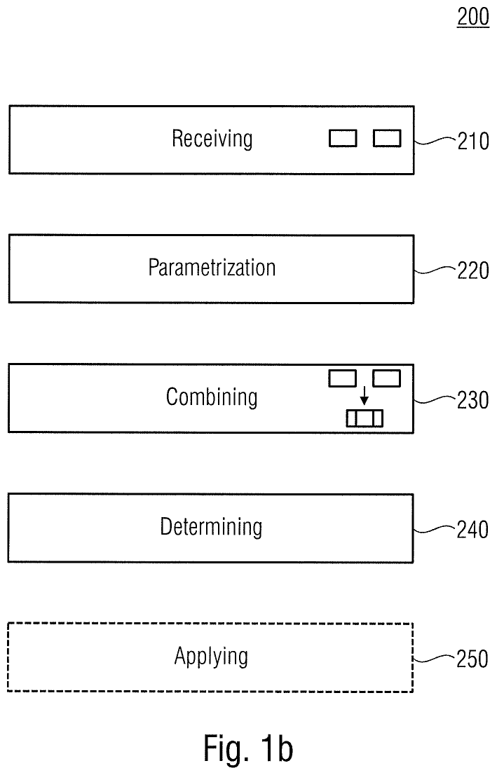

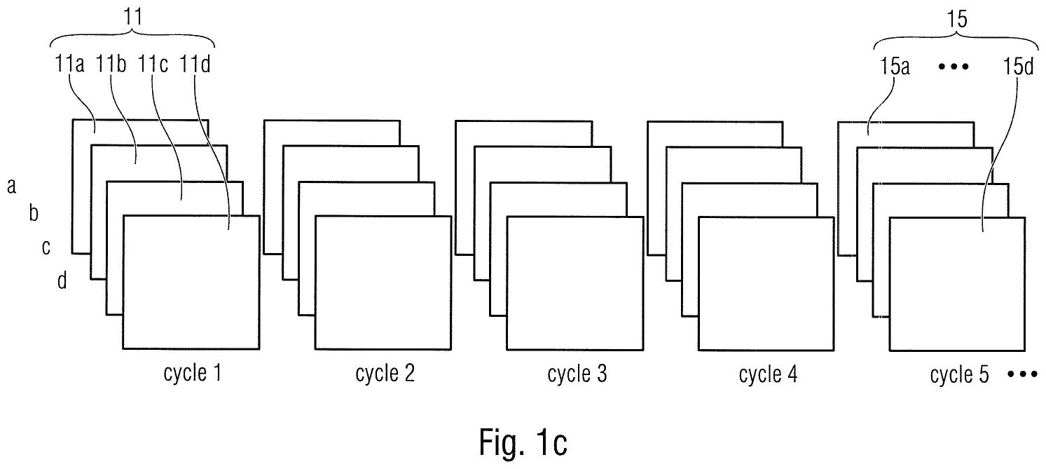

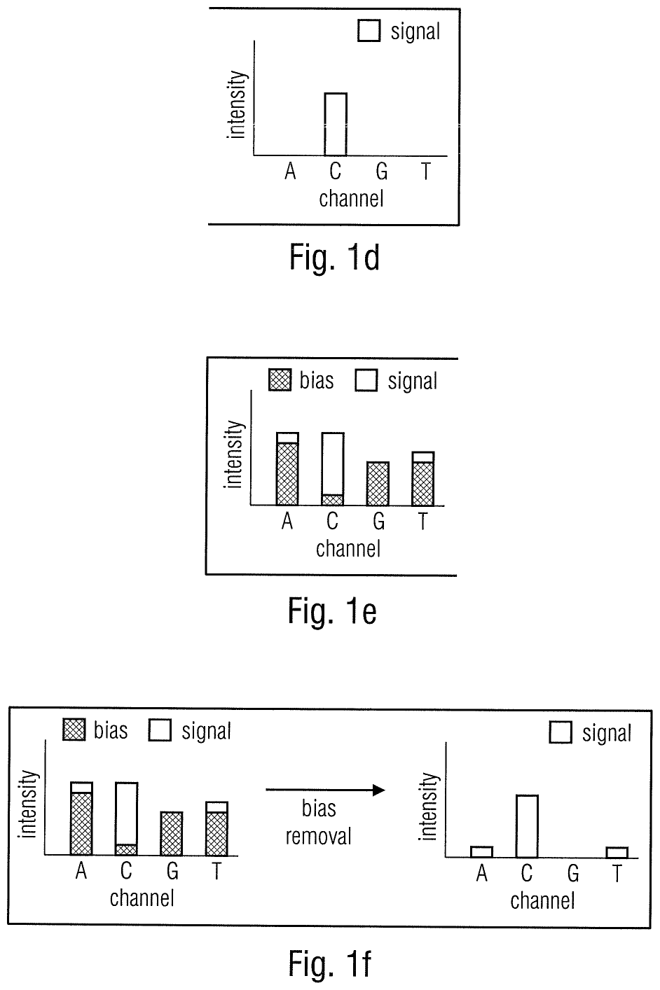

FIG. 1a shows a flowchart illustrating the method for performing a normalization of intensity values belonging to image data used for sequencing analyses, wherein the normalization is performed over a plurality of channels (one cycle) according to an embodiment;

FIG. 1b shows a flowchart illustrating the method for normalization of the intensity values, wherein the normalization is performed over a plurality of cycles according to another embodiment;

FIG. 1c shows a schematic representation of image data belonging to different channels and different cycles for illustrating the principle of sequencing analysis;

FIGS. 1d-1e show schematic diagrams illustrating the intensity differences between the single channels used for the sequencing analysis;

FIG. 1f shows schematic diagrams of intensity distributions for illustrating the improvements achieved by the normalization according to embodiments;

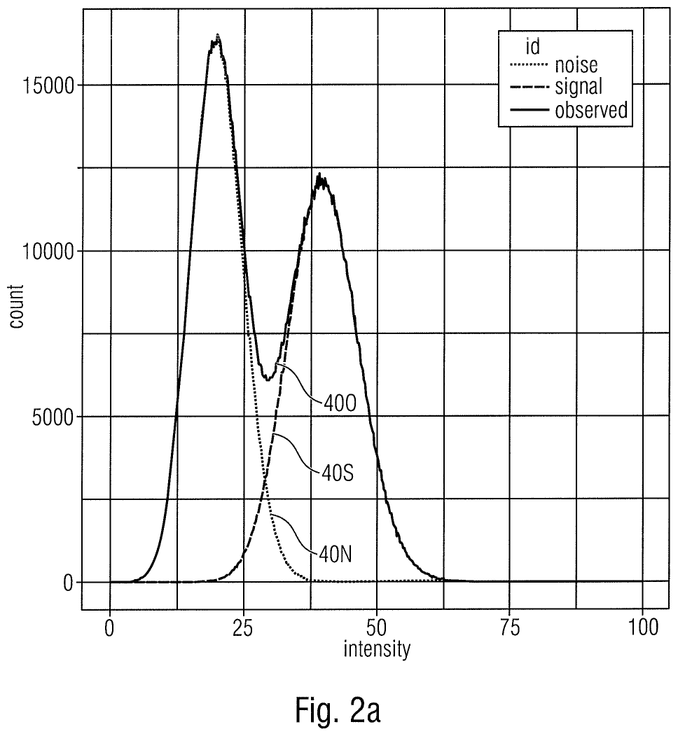

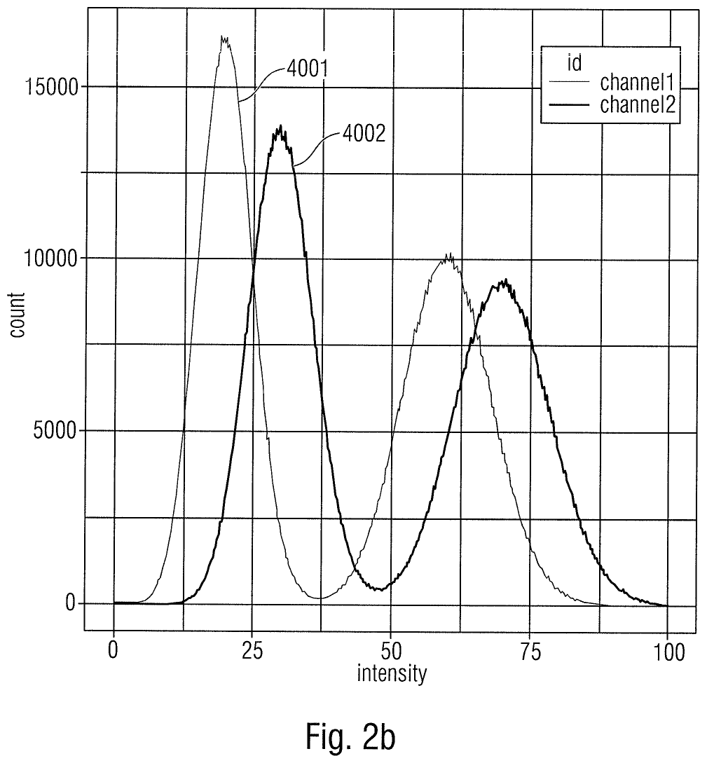

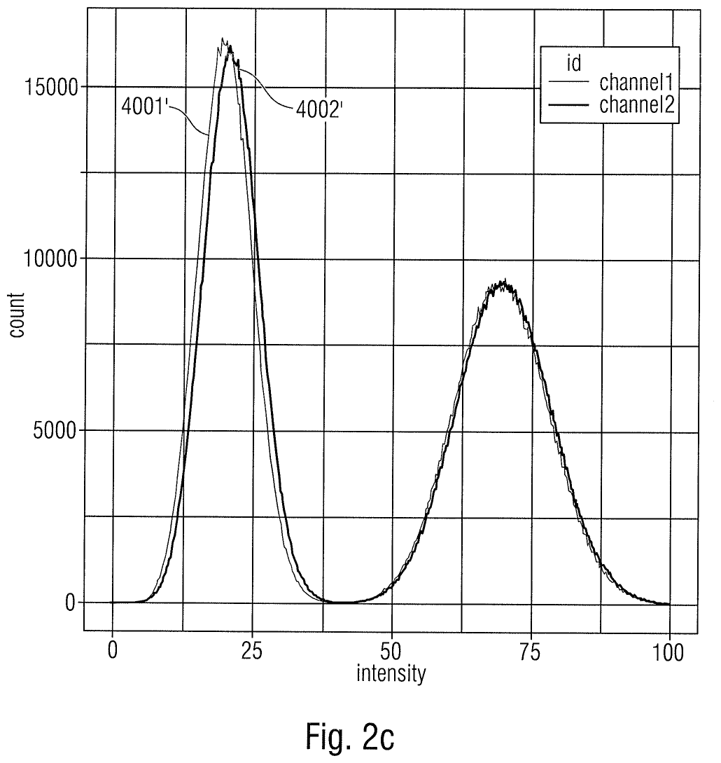

FIGS. 2a-c show schematic diagrams illustrating intensity distributions and normalization thereof according to an embodiment; and

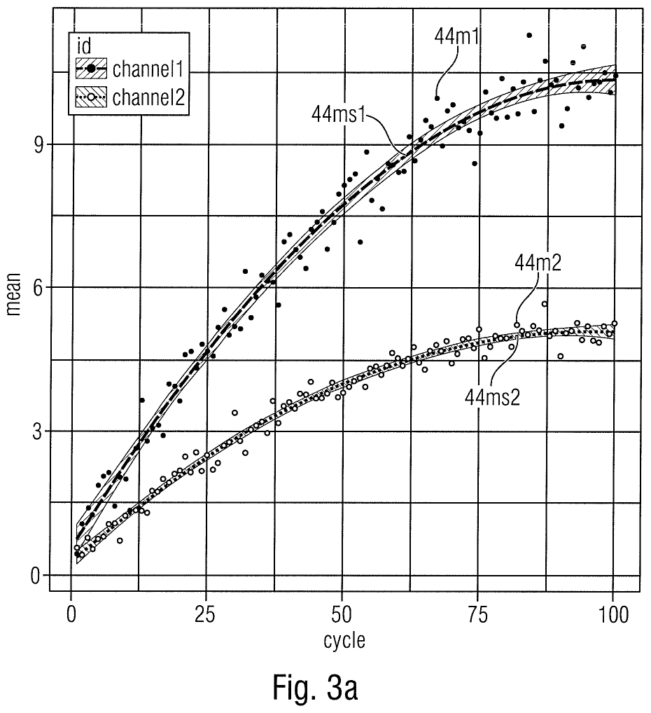

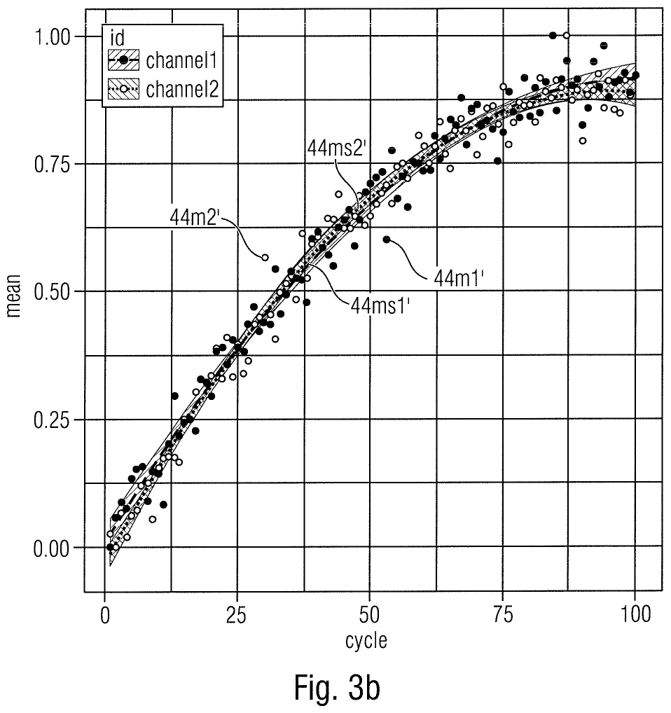

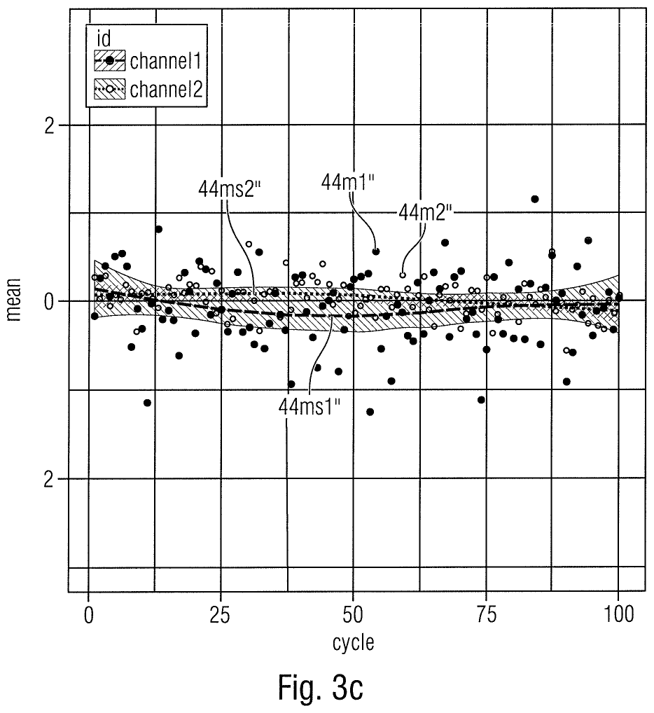

FIGS. 3a-c show schematic diagrams of intensity distribution-characterization-parameter for illustrating the principle of performing the normalization over a plurality of cycles.

Below, embodiments will subsequently be discussed in detail referring to the enclosed figures. Here, identical reference numerals are provided to elements on method steps having similar or identical functions so that the description thereof is mutually applicable and interchangeable.

FIG. 1a shows the basic method 100 for performing a normalization of intensity values. This method 100 is mainly used for sequencing analysis. For sequencing analysis or DNA analysis single-stranded DNA fragments of the template molecules are extended such that the single basis or nucleotides (in general molecules of interest) can be detected using a fluorescent dye.

This is exemplarily illustrated by FIG. 1c. FIG. 1c shows an exemplary set of images generated by a sequencing apparatus. For each sequencing cycle 11 to 15 a set of n images 11a to 11d/11a to 15d is acquired, one for each channel (belonging to specified fluorescent dye). Note that in some images the channels A to D are referred to A, C, G and T, which is the official labelling used for (DNA) sequencing. The intensity values are encoded. While white pixel may represent emitted light by fluorophores, dark pixel areas may represent background intensities.

Typically a distinction is made between four nucleotides (A, C, G and T), such that four distinguishable fluorescent dyes are used. Each channel can be detected using an own channel. The channels can be analyzed using different filter settings, so that a plurality of images 11a to 11d has to be analyzed during one cycle. Each cycle refers to a sequencing iteration (cf. cycle 11, cycle 12, cycle 13, etc., cycle 15, etc.). Background thereof is that during each sequencing iteration/cycle 11 to 15, just one nucleotide can be detected. Just for the sake of completeness, it should be noted that between the single cycles 11 to 15 a procedure comprising cleaving the fluorescent dye and extending the sequencing primer is performed such that a new base/nucleotide can be incorporated.

Since for each sequencing cycle 11 to 15 a set of n images (11a-11d) are required. The entire sequencing run is represented by a set of n×c images 11a to 15d, where n equals the number of channels (typically four) and c equals the number of cycles (here five). Expressed in other words, this means that each sequencing run has two main dimensions, namely the dimension defined by the number of cycles and the dimension defined by the number of channels.

As indicated above, the signal distribution among the channels a-d (A, C, G and T) determines base-calling. In the optimal case just one channel is characterized by non-zero intensity values. This optimal case is shown by FIG. 1d. As illustrated by FIG. 1d just the channel c outputs a signal. In such a case it can be assert that within the examined cycle at the examined position of the image a nucleotide of the type c is detected.

However, in reality the measured intensity signal is distorted by biasing and noise components. Such biased intensity values are illustrated by FIG. 1e. Here, the biasing and the signal component of each signal belonging to a respective channel are marked by different hatchings. Typically, these components lead to a systematic offset from zero within the specific channels. Due to the biasing the intensity values for the channels a and d are nearly the same. Therefore, the two channels cannot easily be distinct from each other.

The method 100 enables to remove the channel-specific contribution to bias and/or noise by performing a normalization. Here, the data driven approach enables to normalize intensity values over all channels A to D (preferably within one cycle 11, 12, etc. or 15). Within the first step 110 the plurality of image data for the plurality of channels, i.e., for the example of FIG. 1C, the images 11a to 11a are received. Each image 11a to 11d describes, for the respective channel A to D, an intensity distribution over all positions of the respective image element 11a to 11d or, at least over a subset of all positions, when just a portion of each image 11a to 11d should be analyzed. After that, all image data for the first cycle 11 are available.

The intensity distributions over the relevant positions of the images 11a to 11d, or of at least for two images, are parametrized in order to obtain a parametrized distribution for the plurality of channels. This step is marked by the reference numeral 120. The parametrized distribution can, for example, be a summary statistic or can be described using a specific parameter, e.g., the mean. Therefore, the step 120 can comprise the sub-step of determining a significant parameter or parameter set describing the behavior of the respective channel, like the summary statistic. Alternatively, a maximum-likelihood estimation or aposteriori-estimation can be used.

Here it should be noted that the intensity distribution over all positions and/or the parametrized distributions describe, for example, the number of counts in correlation to respective intensity values for the respective channel of the plurality of channels a-d (cf. FIG. 2a). Thus, the intensity distribution is defined within a two dimensional space.

Within the next step 120, the plurality of the parametrized distributions for all or at least two channels a to d (A, C, G and T) are combined such that a common distribution for all or the relevant channels within the first cycle can be obtained. For example, the common distribution may be an average of all or all relevant parametrized distributions.

In context of the common distribution it should be noted that the common distribution (for at least two, all relevant or all channels a-d) represents an average of (at least two) all relevant (or all) parametrized distributions/intensity distributions of the plurality of channels. For example, the common distribution for (at least two) all relevant (or all) channels may describe an averaged number of counts in correlation to an averaged intensity values for at least two, all relevant or all channels a-d (cf. FIG. 2c). Consequently, the common distribution or each point within the common distribution may be defined by at least two parameters (number of counts and intensity), e.g. derived from the parametrized distributions. Consequently, the common distribution is also defined within a two dimensional space.

Starting from the common distribution, the intensity values of the respective channels 11a to 11d can be mapped to the common distribution using a respective transfer function for each channel 11a to 11d. This step is marked by the reference numeral 140. The respective transfer functions can be used for filtering or normalizing the images 11a to 11c. Since the respective transfer function is determined based on all or at least all relevant positions (a sub-set of all positions) of the respective channel 11a to 11d, the transfer function enables the channels 11a to 11d to have—an averaged—same behavior, so that channel specific effects can be avoided or eliminated.

Example: starting from a basic example, merely that one channel has a substantially higher brightness than another channel it is clear that the determined transfer function enables dimming of the entire bright channel, especially, each intensity value of the single position within the channel. Due to the dimming (application of the channel specific transfer function, the intensity values belonging to the signal positions within the channels are more comparable than without applying the transfer function or normalization procedure.

If now, the image analysis of the respective channel 11a to 11d or, in more detail, of each relevant position (sub-set of all positions) or at least of one position of the respective channel 11a to 11d, is performed while applying the respective transfer function to the intensity value, the intensity values of the different channels 11a to 11d are distinguishable from each other. This is illustrated by FIG. 1f.

FIG. 1f shows in the left-hand diagram the measured intensity values of FIG. 1f. The application of the respective transfer function to the respective channel enables removal of the bias, such that an intensity values (for the relevant positions/the one position) within the four channels can be achieved, which approximately equates to the intensity diagram as discussed with respect to FIG. 1d. Here, it is important that this bias cannot be extracted directly from the base call but has to be estimated via the intensity value distributions over all base calls as described above. Due to the application of the transfer function, the channel specific biases are removed by filter normalization and, allowing unambiguous base-calling to channel C.

The application of the channel specific transfer function to the intensity values is an optional step which is marked by the reference numeral 150. The usage of the method steps 110 to 140 enables determination of the transfer functions, wherein the performing of all method steps 110 to 150 enables the normalization of the channels within one cycle. Therefore, starting from a basic approach, the step 150 is an optional step.

Although in embodiments the normalization has been described in context of normalizing different channels to each other, the normalization can also additionally or alternatively be performed such that one or more channels are normalized over the plurality of cycles. This approach is illustrated by FIG. 1B showing a block diagram of method 200. Method 200 comprises the basic steps 210 to 240 and the optional step 250. The step 210 is comparable to the step 110, wherein the image data does not only comprise the images of one cycle 11, but also the images belonging to a plurality of cycles or all cycles. Within the step 220 the intensity distributions over all positions or over all relevant positions of the received images are parametrized. After that, the parametrized distributions, at least for one channel over the plurality of cycles, are combined in order to obtain a common distribution for at least one channel over the plurality of cycles. The step is marked by the reference numeral 230. This step may optionally be performed such that a common distribution for plurality of cycles and channels is obtained. Starting from the common distribution, the determining of the respective transfer function for the at least plurality of cycles 11 to 15 is performed within the step 240. The step 250 is the optional step of applying the determine transfer functions during the image analysis.

With respect to FIGS. 2A and 2C, the background of the above method will be described.

FIG. 2A shows a diagram illustrating the number of counts (y axis) for respective intensity values (plotted over the x axis). Here, the observed values are marked by the reference numeral 40O. This observed signal 40O results from two signal portions, namely the noise 40N and the real signal 40S. This schematic representation of the intensity distribution for a given channel over all positions makes it clear that the observed intensity distribution 40O is the result of positions with intensities drawn from the noise distribution 40N and those drawn from the signal distribution 40S. Dependent on the separation of noise and signal distribution, the observed distribution may or may not be bimodal. The diagram can be described with other words, in that the intensity values 40O are observed in a given channel can be modelled by a random variable. For each position the random variable is drawn from one of two distinct distributions, namely “signal distribution” 40S in case the channel indicates an incorporated nucleotide match and, “noise distribution” 40N, otherwise. The observed intensity-distribution over all positions will be characterized by the combination of both distributions. The intensity distribution may be channel-specific, leading to systematic differences as can be seen by FIG. 2B.

FIG. 2B shows a schematic representation of intensity distributions for two channels 40O1 and 40O2. Here, the observed intensity distributions 40O1 and 40O2 are distinct for both channels. This difference may lead to biased-based calling.

By the usage of the channel specific transfer functions, the observed intensities over all positions 40O1 and 40O2 can be distorted such that the same follow a common distribution. This means that the goal of the approach is to collapse all channel specific intensity distributions into one common distribution, thereby producing unbiased intensity profiles over all channels, leaving two unbiased-base calling. This collapse may be done via means of the channel specific transfer functions determined using the method 100. The result is shown by FIG. 2C.

FIG. 2C shows a schematic representation of corrected intensity distribution for two channels (corresponding to the common distribution). Here, the corrected intensity diagrams are marked by the reference numeral 40O1′ and 40O2′. This collapse of the channel specific intensity distributions 40O1′ and 40O2′ may lead to unbiased-base calling.

Here, the observed intensity values of all positions within the respective channels are distorted using the transfer function, such that the intensity distributions 40O1′ and 40O2′ are achieved.

Starting from the normalization between two or more channels, a preferred way to determine the respective transfer functions enabling collapse of all channel-specific intensity distribution into one distribution will be discussed. For this, the observed intensity distributions have to be characterized.

In order to collapse all channel specific intensity distributions into one common distribution, the observed intensity-distributions have to be characterized. Distribution characterization can be classified into two approaches: i) parametrization of a defined distribution and ii) characterization of an undefined distribution. The first case applies if the phenomena leading to noise and signal are known and the resulting family of observed distribution can be derived. In this case parametrization can be performed by probabilistic modeling and standard procedures like maximum-likelihood-estimation or maximum-aposteriori-estimation. If no probabilistic model of the observed intensity distribution is known, characterization may occur via summary statistics. Depending on the complexity of the underlying distribution and how distinct the channel specific distributions are this can be performed by a single summary statistic or a combination of summary statistics. Common applicable summary statistics include mean, mode, standard deviation (SD) as well as order statistics.

Given the set of characterized intensity distributions for all channels, the second step is to find a common distribution that all channel-specific distributions can be collapsed to. This is performed by channel-specific transformations of the data such that the resulting intensity distributions follow a distribution characterized by specified parameters or summary statistics. The nature of the transformation depends on the underlying distribution and may be a simple linear transformation, a non-linear transformation, a set of linear or non-linear transformations or a combination of both sets. In the simplest case, intensity distribution may be described by a Gaussian distribution. In this case channel specific distributions can be collapsed by normalizing the Gaussian distribution by subtraction of the mean and division by SD.

Analogously, instead or additionally, to the intensity distribution normalization between plurality of channels as cycle-specific normalization can be used.

The intensity normalization approach described above may be applied to all cycles or for each cycle individually. The latter case may be desirable to correct for differences introduced as a function of sequencing cycle (i.e. drift). Drift may occur for example in the case of cycle specific sequencing configuration (i.e. adapted imaging or chemistry) or due to decaying or increasing performance. The cycle-specific normalization can be performed as described above. Alternatively, the cycle context can be used to smooth normalization transformations. This case depends on the assumption that channel-specific distributions of subsequent cycles are similar and thus their characterization parameters (i.e. distribution parameters or summary statistics) are correlated. For smoothing, channel specific distribution characterization is performed as described above for each cycle or groups of cycles. Smoothing may be performed by a variety of functions including sliding window approaches (mean, median, Gaussian filter, local model fitting) or model fitting on the entire cycle set (e.g. polynomials). To estimate the transformation model to the specified target distribution, the parameter estimated by the smoothing function is used instead of the parameter derived from the distribution characterization.

This approach will be discussed with respect to FIGS. 3A to 3C.

FIG. 3A shows a schematic representation of one distribution-characterization-parameter, here, the mean. The mean, as an example of a summary statistic, can be used to characterize the intensity distribution for the two channels. This parameter for the two channels is marked by the reference numeral 44M1 and 44M2. The mean is determined for each cycle individually, as indicated by the dots, and plotted versus the cycle number. As can be seen, the subsequent means are correlated, but show significant noise. The noise can be removed which results in the smoothed line. After noise removal, the mean retains significant differences between both channels indicating a systematic difference which may lead to biased-base calling. The smoothed line may then be used instead of the noisy cycle-specific values, e.g. for the channel specific normalization.

In order to eliminate the systematic differences, the distributions can be normalized using the above-discussed approach according to which the plurality of channels is distorted using the transfer functions such that each channel is mapped to a common distribution. The transformation can be performed such that the general trend of the parameter versus cycle is retained.

This approach is illustrated by FIG. 3B showing the distorted means 44M1′ and 44M2′ of the two channels together with the smoothed means 44MS1′ and 44MS2′. The distorted values 44M1′ and 44M2′ and 44MS1′ and 44MS2′ leave the overall trend of the mean with respect to the cycle intact, but normalizes the distributions for each cycle such that the mean is similar for both channels. Here, the target distribution is selected such that the mean is scaled between 0 and 1. This can be achieved by the definition of the target distribution and the derivation of the transformation to reach this distribution. If the general trend should be retained the defined target distribution should reflect this. If the trend is the same, for all channels collapsing can be achieved by normalizing the characterization parameters between defined values as shown here (min=0, max=1).

Expressed in other words, this means that according to an embodiment, a normalization between the single channels together with a smoothing can be used. Due to the interchannel-normalization, the distinction between the same is improved wherein the smoothing enables avoidance of jumps between subsequent cycles.

According to another embodiment, a further approach normalizes both the channels with respect to the other channels and the radius over all cycles such that the parameter profiles are stationary with respect to the plurality of cycles. This approach is illustrated by FIG. 3C. FIG. 3C shows the means for the two channels 44M1″ and 44M2″ together with the smoothed versions thereof, 44MS1″ and 44MS2″. As can be seen, this second approach enables generation of stationary distributions over the entire sequencing run. This is achieved by the definition of the target distribution which should be identical for all cycles. The obtained transfer function for the respective channel can be used to normalize the channels during/for performing the sequencing analysis.

Below, an enhanced example will be discussed. Let us assume that we measure intensities in 4 distinct channels and that for each cycle the channel-specific intensity values are distributed according to a Gaussian distribution with distinct mean, but identical SD. In this case the intensity-distribution normalization is simple: i) The channel-specific distribution for each cycle is characterized by the mean of the intensities. ii) Mean versus cycle is determined for each channel individually and smoothed using an appropriate method (e.g. windowed mean). iii) A target distribution for each cycle is determined. Let us further assume that the mean of intensities increases with respect to cycles with a similar function for all channels and that we want to retain this general trend. To retain the mean versus cycle trend, the mean of the target distribution is specified such that it ranges from 0 to 1 and follows the overall trend. iv) Transformation is performed by subtracting the smoothed observed channel mean and adding the mean of the target distribution.

Other examples of collapsing channel intensity distribution may include normalizing Gaussian distributions with distinct mean and standard deviation via standardization (i.e. mean=0, SD=1) or transformation of non-normal distribution to approximate normal distributions (e.g. log-transformation, square-root transformation, Box-Cox transformation, Yeo-Johnson transformation) followed by the standardization of the resulting approximately normal distribution.

Although, referring to the above discussion, all embodiments have been described in the context of a method it should be noted that the idea may also be implemented as an apparatus. Here, all the above implementation aspects may also be used in context of the apparatus.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a digital storage medium, for example a floppy disk, a DVD, a Blu-Ray, a CD, a ROM, a PROM, an EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may for example be stored on a machine readable carrier.

Other embodiments comprise the computer program for performing one of the methods described herein, stored on a machine readable carrier.

In other words, an embodiment of the inventive method is, therefore, a computer program having a program code for performing one of the methods described herein, when the computer program runs on a computer.

A further embodiment of the inventive methods is, therefore, a data carrier (or a digital storage medium, or a computer-readable medium) comprising, recorded thereon, the computer program for performing one of the methods described herein. The data carrier, the digital storage medium or the recorded medium are typically tangible and/or non-transitionary.

A further embodiment of the inventive method is, therefore, a data stream or a sequence of signals representing the computer program for performing one of the methods described herein. The data stream or the sequence of signals may for example be configured to be transferred via a data communication connection, for example via the Internet.

A further embodiment comprises a processing means, for example a computer, or a programmable logic device, configured to or adapted to perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

The above described embodiments are merely illustrative for the principles of the present invention. It is understood that modifications and variations of the arrangements and the details described herein will be apparent to others skilled in the art. It is the intent, therefore, to be limited only by the scope of the impending patent claims and not by the specific details presented by way of description and explanation of the embodiments herein.

1. A method for performing a normalization of intensity values obtained to perform sequencing analysis, comprising:
receiving a plurality of image data for a plurality of channels, wherein each image data of the plurality of image data describes for a respective channel of the plurality of channels an intensity distribution over all positions of an image of the plurality of image data;
parameterizing the intensity distribution of each respective channel of the plurality of channels over all the positions of the image to obtain parametrized distributions for the plurality of channels;
combining the parametrized distributions for the plurality of channels to obtain a common distribution for all of the plurality of channels; and
determining, for each of the plurality of channels, a respective transfer function such that the respective transfer function for the respective channel maps the corresponding intensity distribution to the common distribution.

2. The method of claim 1, wherein the parameterizing and the determining are performed for four channels, each belonging to a specific nucleotide, or
wherein the parameterizing and the determining are performed for four channels, wherein each of the four channels is filtered to determine one of four distinct fluorescent dyes marking four specific nucleotides.

3. The method of claim 1, wherein each position within the plurality of channels belongs to its own sequence.

4. The method of claim 1, wherein the parameterizing is performed using a substep of maximum-likelihood-estimation, maximum-aposteriori-estimation, determining a summary statistic for one or more channels of the plurality of channels, or determining a specified parameter set describing the intensity distribution for a respective channel of the plurality of channels.

5. The method of claim 1, wherein the respective transfer function enables a linear transformation of the corresponding intensity distribution to the common distribution.

6. The method of claim 1, wherein the respective transfer function enables a non-linear transformation of the corresponding intensity distribution to the common distribution.

7. The method of claim 1, wherein the intensity distribution for a respective channel of the plurality of channels is described by a Gaussian distribution with a distinct mean and a distinct standard deviation.

8. The method of claim 7, wherein the respective transfer function for the respective channel described by the Gaussian distribution comprises mathematic operations of subtracting a mean from the corresponding intensity distribution or dividing the corresponding intensity distribution by a standard deviation.

9. The method of claim 6, wherein the intensity distribution for a respective channel of the plurality of channels is described by a non-normal distribution.

10. The method of claim 9, wherein the respective transfer function enables a log-transformation, a square-root transformation, a Box-Cox transformation, or a Yeo-Johnson transformation.

11. The method of claim 1, wherein the parameterizing, the combining, and the determining are performed for each cycle individually.

12. The method of claim 1, wherein the parameterizing, the combining, and the determining are performed for a plurality of cycles or all cycles.

13. The method of claim 12, wherein the the respective transfer function for the respective channel comprises a smoothing function, and
wherein the smoothing function describes a maximum change of an intensity value over the plurality of cycles.

14. The method of claim 12, wherein each respective transfer function comprises a respective normalization function, and
wherein the respective normalization function is determined such that intensity values of the plurality of channels are normalized over the plurality of cycles for all channels.

15. The method of claim 14, wherein the respective normalization function is determined if no trend of one or more channels over the plurality of cycles is detected.

16. The method of claim 12, wherein the common distribution for all of the plurality of channels is described by a set of functions comprising for each channel a common distribution function, wherein at least two of the common distribution functions differ from each other and
wherein each respective transfer function is determined such that the respective transfer function for the respective channel maps the corresponding intensity distribution to the corresponding common distribution function.

17. The method of claim 15, wherein the plurality of common distribution functions is determined if a trend of one or more channels over the plurality of cycles is detected.

18. The method of claim 16, wherein each respective transfer function comprises a respective smoothing function, wherein the respective smoothing function describes a maximum change of an intensity value over the plurality of cycles, or
wherein the respective smoothing function is determined such that the intensity value used for the respective channel is smoothed over the plurality of cycles.

19. The method of claim 1, wherein the intensity distribution over all positions or the parametrized distributions describe a number of counts in correlation to respective intensity values for the respective channel of the plurality of channels, or
wherein the common distribution for at least two channels represents an average of at least two parametrized distributions of the plurality of channels, or
wherein the common distribution for at least two channels describes an averaged number of counts in correlation to an averaged intensity values for at least two channels.

20. A non-transitory computer readable storage medium having instructions stored thereon that, when executed by at least one computing device, cause the at least one computing device to perform operations comprising:
receiving a plurality of image data for a plurality of channels, wherein each image data of the plurality of image data describes for a respective channel an intensity distribution over all positions of an image of the plurality of image data;
parameterizing the intensity distribution of each respective channel of the plurality of channels over all the positions of the image to obtain parametrized distributions for the plurality of channels;
combining the parametrized distributions for the plurality of channels to obtain a common distribution for all of the plurality of channels; and
determining, for each of the plurality of channels, a respective transfer function such that the respective transfer function for the respective channel maps the corresponding intensity distribution to the common distribution.

21. A system performing a normalization of intensity values obtained to perform sequencing analysis, comprising:
an interface configured to:
receive a plurality of image data for a plurality of channels (a-d), wherein each image data of the plurality of image data describes for a respective channel of the plurality of channels an intensity distribution over all positions of an image of the plurality of image data; and
a processor configured to:
parametrize the intensity distribution of each respective channel of the plurality of channels over all the positions of the image to obtain parametrized distribution for the plurality of channels;
combine the parametrized distribution for the plurality of channels to obtain a common distribution for all of the plurality of channels; and
determine, for each of the plurality of channels, a respective transfer function such that the respective transfer function for the respective channel maps the corresponding intensity distribution to the common distribution.
